(19) United States
(12) Patent Application Publication
(10) Pub. No.: US 2022/0142563 A1
(43) Pub. Date: May 12, 2022

(54) SYSTEMS AND METHODS FOR SLEEP DETECTION

(71) Applicant: Eli Lilly and Company, Indianapolis, IN (US)

(72) Inventors: Haoda FU, Carmel, IN (US); Zhao LU, Zionsville, IN (US); Jian WANG, Carmel, IN (US)

(21) Appl. No.: 17/438,150

(22) PCT Filed: Mar. 25, 2021
(86) PCT No.: PCT/US2021/024088

§ 371 Date: Sep. 10, 2021

Related U.S. Application Data
(60) Provisional application No. 63/004,077, filed on Apr. 2, 2020.

(51) Int. Cl. A61B 5/00; A61B 5/11; G06N 20/10

(52) U.S. Cl. CPC A61B 5/4809; A61B 5/11; G06N 20/10; A61B 5/681; A61B 5/7267

(57) ABSTRACT

Methods and systems are provided for determining when a subject is awake or asleep based on movement data derived from a movement sensor worn by the subject during a monitoring period. The methods/systems comprise processing the movement data using a linear programming support vector regression (LP-SVR) algorithm to derive a set of support vectors, analyzing the support vectors to determine a first set of time periods within which a measure of density over time of the support vectors is below a predetermined threshold, and to determine a second set of set of time periods within the monitoring period within which the measure of density over time of the support vectors is above the predetermined threshold. The first set of time periods are when the subject is likely to be asleep, while the second set of time periods are when the subject is likely to be awake.

### FIELD OF THE DISCLOSURE

The present disclosure relates to devices and methods for determining when a subject is awake and when the subject is asleep. More particularly, the present disclosure relates to determining when the subject is awake or asleep based on time-series movement data recorded by a movement sensor worn by the subject.

### BACKGROUND OF THE DISCLOSURE

Physical activity and sleep are both recognized as important health determinants and may prevent or mitigate chronic or neurologic diseases. Hence, the monitoring of sleep by quantifying sleep time and quality is important in many preventative health care scenarios. Many research and clinical situations call for an inexpensive and unobtrusive method of measuring human sleep/wake time outside of the laboratory.

For these reasons, characterization of sleep via wrist actigraphy has gained popularity in clinical and research settings as an alternative to polysomnography (PSG). Though considered an accurate measure for sleep assessment, PSG can be costly, labor-intensive, and invasive to implement. Also, PSG typically involves sleeping in a clinical or laboratory setting away from the subjects' homes and can only be reasonably implemented for 1-2 nights at a time. Advantages of actigraphy include its low cost, convenience, and ability to objectively estimate sleep in large populations for periods of up to months at a time.

Many existing unsupervised sleep/awake detection algorithms using actigraphy rely on activity counts over pre-defined time spans. Time periods during which activity counts exceed a certain pre-determined threshold are considered time periods during which the subject is awake. Conversely, time periods during which activity counts are below the pre-determined threshold are considered time periods during which the subject is asleep. However, it can be difficult to define a threshold that accurately distinguishes between awake/asleep time periods for all or most subjects in a population, as some subjects move a lot while asleep and other subjects remain quite still even while awake. Therefore, a threshold that works for one subject may not work as well for other subjects in distinguishing between awake/asleep time periods.

Therefore, a need exists for methods and devices to distinguish between awake/asleep periods based on movement data without relying solely on activity counts.

### SUMMARY

According to a first exemplary aspect of the present disclosure, a computer-implemented method is provided for determining when a subject is awake and when the subject is asleep, the method comprising: receiving, at at least one processor, time-series movement data for the subject derived from accelerometer data recorded by a movement sensor worn by the subject during a monitoring period, wherein the movement data indicates at least a magnitude of bodily movement of the subject at multiple times during the monitoring period; processing, by the at least one processor, the movement data using a linear programming support vector regression (LP-SVR) algorithm to derive a set of support vectors, wherein each support vector corresponds to a different time point during the monitoring period; analyzing, by the at least one processor, the derived set of support vectors to determine a first set of time periods within the monitoring period within which a measure of density over time of the support vectors is below a predetermined threshold, and to determine a second set of time periods within the monitoring period within which the measure of density over time of the support vectors is above the predetermined threshold; and outputting, by the at least one processor, at least one of (i) the first set of time periods as periods during which the subject is likely to be asleep, and (ii) the second set of time periods as periods during which the subject is likely to be awake.

In some embodiments, the measure of density over time of the support vectors comprises a count of the number of support vectors within a moving time window having a predetermined duration.

In some embodiments, the movement data is derived from the accelerometer data by passing the accelerometer data through at least one of a low-pass filter, a high-pass filter, a band-pass filter, and a moving average smoothing filter.

In some embodiments, the movement sensor comprises a wrist-mounted actigraph sensor.

In some embodiments, the at least one processor outputs the at least one of the first set of time periods and the second set of time periods to a user-interface displayed on a visual display.

According to a second exemplary aspect of the present disclosure, a system is provided for determining when a subject is awake and when the subject is asleep, the system comprising: a movement sensor configured to be worn by the subject during a monitoring period and to record accelerometer data indicating at least a magnitude of bodily movement of the subject at multiple times during the monitoring period; and a computing device comprising: at least one communication device configured to receive the recorded accelerometer data from the movement sensor, and at least one processor configured to: process the recorded accelerometer data to derive time-series movement data for the subject; process the movement data using a linear programming support vector regression (LP-SVR) algorithm to derive a set of support vectors, wherein each support vector corresponds to a different time point during the monitoring period; analyze the derived set of support vectors to determine a first set of time periods within the monitoring period within which a measure of density over time of the support vectors is below a predetermined threshold, and to determine a second set of time periods within the monitoring period within which the measure of density over time of the support vectors is above the predetermined threshold; and output at least one of (i) the first set of time periods as periods during which the subject is likely to be asleep, and (ii) the second set of time periods as periods during which the subject is likely to be awake.

According to a third exemplary aspect of the present disclosure, non-transitory computer-readable media storing computer executable instructions for determining when a subject is awake and when the subject is asleep is provided. The instructions, when executed by at least one processor, is operable to cause the at least one processor to: receive time-series movement data for the subject derived from accelerometer data recorded by a movement sensor worn by the subject during a monitoring period, wherein the movement data indicates at least a magnitude of bodily movement of the subject at multiple times during the monitoring period; process the movement data using a linear programming support vector regression (LP-SVR) algorithm to derive a set of support vectors, wherein each support vector corresponds to a different time point during the monitoring period; analyze the derived set of support vectors to determine a first set of time periods within the monitoring period within which a measure of density over time of the support vectors is below a predetermined threshold, and to determine a second set of time periods within the monitoring period within which the measure of density over time of the support vectors is above the predetermined threshold; and output at least one of (i) the first set of time periods as periods during which the subject is likely to be asleep, and (ii) the second set of time periods as periods during which the subject is likely to be awake.

### BRIEF DESCRIPTION OF THE DRAWINGS

The above-mentioned and other features and advantages of this disclosure, and the manner of attaining them, will become more apparent and will be better understood by reference to the following description of embodiments of the invention taken in conjunction with the accompanying drawings, wherein:

FIG. 1 is a block diagram illustrating the main components of an exemplary system for detecting sleep/awake periods in a subject.

FIG. 2 is a flowchart depicting an exemplary method for determining sleep/awake periods during a monitoring period for a subject based on time-series movement data.

FIG. 3 is a flowchart depicting an exemplary method for preprocessing raw accelerometer data to derive the time-series movement data.

FIG. 4, FIG. 5, and FIG. 6 show exemplary results obtained by the disclosed logic when applied to a publicly available benchmark dataset.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

The term “logic” or “control logic”, as used herein may include software and/or firmware (also referred to herein as a “method”, “process”, or “application”) executing on one or more programmable processors, application-specific integrated circuits (ASICs), field-programmable gate arrays (FPGAs), digital signal processors (DSPs), hardwired logic, or combinations thereof. Therefore, in accordance with the embodiments, various logic may be implemented in any appropriate fashion and would remain in accordance with the embodiments herein disclosed.

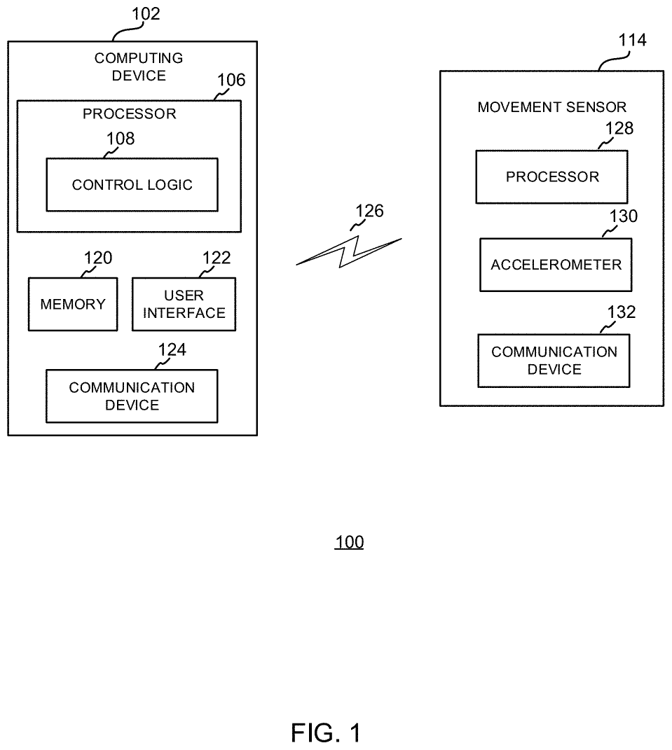

FIG. 1 is a block diagram illustrating the main components of an exemplary system 100 for detecting sleep/awake periods in a subject. System 100 comprises a computing device 102 and a movement sensor 114 that are in communication with one another via a communication link 126.

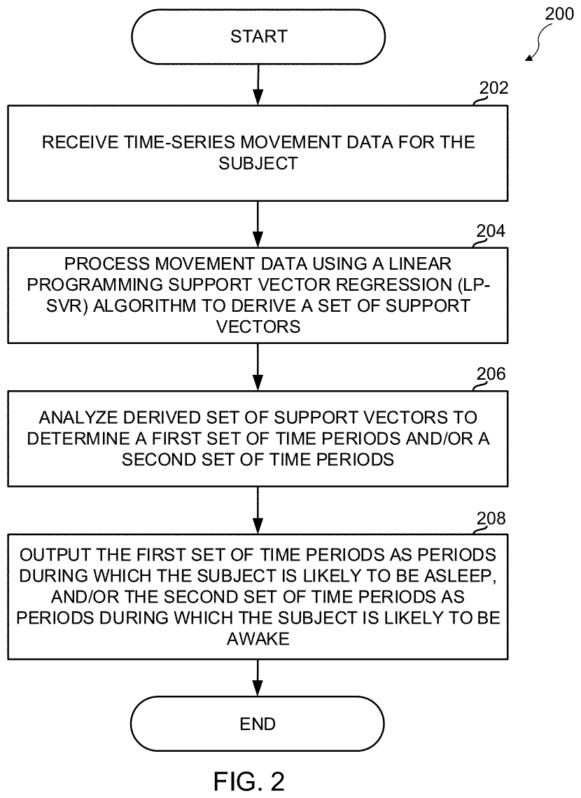

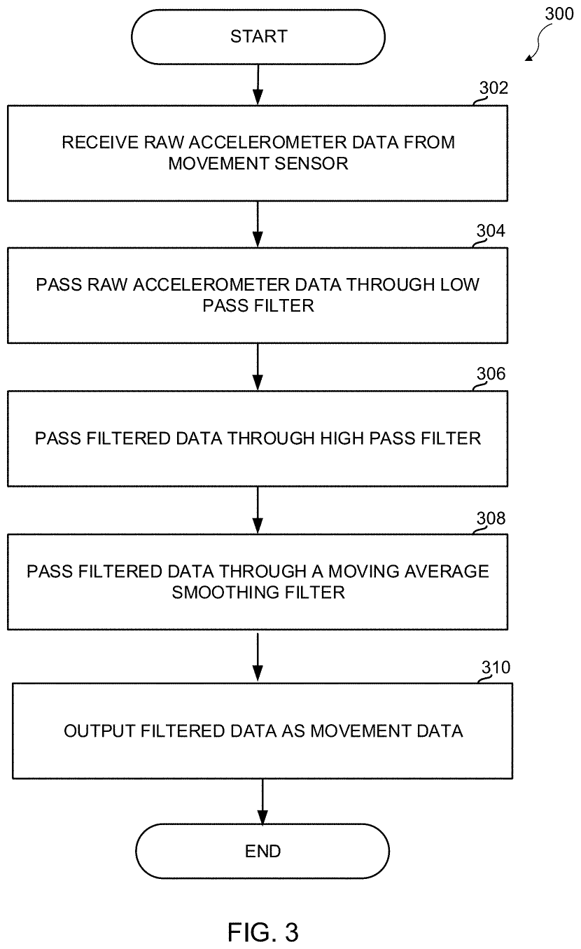

Computing device 102 illustratively includes a mobile device, such as a smartphone. Alternatively, any suitable computing device may be used, including but not limited to a laptop, desktop, tablet, or server computer, for example. Computing device 102 includes at least one processor 106 having sleep detection logic 108 executing software/firmware stored in memory 120 of device 102. The software/firmware code contains instructions that, when executed by sleep detection logic 108 of processor 106, causes computing device 102 to perform the functions described herein, including the methods of FIGS. 2 and 3. The control logic 108 is operative to receive movement data pertaining to a subject, and to process the received movement data to determine time periods during which the subject is awake and time periods during which the subject is asleep. Memory 120 is any suitable computer readable medium that is accessible by processor 106. Memory 120 may be a single storage device or multiple storage devices, may be located internally or externally to processor 106, and may include both volatile and non-volatile media. Exemplary memory 120 includes random-access memory (RAM), read-only memory (ROM), electrically erasable programmable ROM (EEPROM), flash memory, a magnetic storage device, optical disk storage, or any other suitable medium which is configured to store data and which is accessible by processor 106.

Computing device 102 may also optionally include a user interface 122 in communication with processor 102 and operative to provide user input data to the system and to receive and display data, information, and prompts generated by the system. User interface 122 includes at least one input device for receiving user input and providing the user input to the system. In some embodiments, user interface 122 may comprise a graphical user interface (GUI) including a touchscreen display operative to display data and receive user inputs. The touchscreen display allows the user to interact with presented information, menus, buttons, and other data to receive information from the system and to provide user input into the system. Alternatively, a keyboard, keypad, microphone, mouse pointer, or other suitable user input device may be provided.

Computing device 102 further comprises a communication device 124. The communication device 124 can be a wired communication unit (e.g., Universal Serial Bus (USB), FireWire, Serial, etc.) and/or a wireless communication unit (e.g., Bluetooth, WiFi, near-field communication (NFC), and/or the like). For example, the communication device 124 can be a WiFi transceiver, a Bluetooth transceiver, an RFID transceiver, a USB transceiver, a near-field communication (NFC) transceiver, a combination chip, and/or the like. The at least one processor 106 can be configured to communicate with movement sensor 114 using communication device 124, over communication link 126.

Movement sensor 114 illustratively includes a smartwatch or other wrist-mounted actigraph sensor designed to be worn by the subject. In other embodiments, movement sensor 114 may be designed to be worn on other parts of the subject's body. In yet other embodiments, movement sensor 114 may not be designed to be worn by the subject, but may be attached to, placed within, or placed under the subject's bed. In each of these embodiments, movement sensor 114 may be configured to detect, measure, record, and/or transmit information regarding the subject's bodily movement during a monitoring period (e.g., overnight).

Movement sensor 114 comprises at least one processor 128, an accelerometer 130, and a communication device 132. The processor 128 may be configured to control the accelerometer (which may be a two- or three-dimensional accelerometer) to detect, measure, and/or record the subject's bodily movement during the monitoring period. The processor 128 may also be configured to operate communication device 132 to receive instructions and/or data from computing device 102, and/or to send data regarding the subject's bodily movement to computing device 102. The communication device 132 may take the form of any of the embodiments discussed above for communication device 124.

FIG. 2 is a flowchart depicting an exemplary method 200 executed by sleep detection logic 108 of computing device 102 for determining sleep periods and awake periods during a monitoring period for a subject. At step 202, logic 108 receives time-series movement data for the subject. The time-series movement data may be derived from accelerometer data recorded by a movement sensor worn by the subject during a monitoring period, wherein the movement data indicates at least an amount or magnitude of bodily movement observed from the subject at multiple times during a monitoring period. For example, the time-series movement data may be derived from processing raw accelerometer actigraphy data recorded by wearable sensor 114—an exemplary method for processing raw accelerometer data into time-series movement data is described below in FIG. 3. The monitoring period may be a number of hours during which the subject is expected to sleep, e.g., overnight from the hours of 9 pm to 7 am, although any suitable monitoring period may be provided. The time-series data may comprise a magnitude of detected movement or acceleration along one-dimensional axis measured at a particular frequency. In some embodiments, the time-series data may comprise a magnitude of detected movement or acceleration along two- or three dimensions. The time-series data and/or raw accelerometer actigraphy data may be received by processor 106 from movement sensor 114 via communication link 126.

At step 204, logic 108 processes the received time-series data using a linear programming support vector regression (LP-SVR) algorithm to derive a set of support vectors, wherein each support vector corresponds to a different time point during the monitoring period. The LP-SVR algorithm may be considered a non-parametric sparse modelling method, wherein the most informative data points are selected as support vectors for representing the underlying model in a compact form. The presently disclosed system uses LP-SVR as a novel data analysis tool to analyze variability in a time sequence of physiological (e.g., movement) data and recognizing changes in a person's status (e.g., whether the person is awake or asleep) based on that analysis. For the time series analyzed, the support vectors selected by logic 108 are those time instants critical to the variability of the sequence. An exemplary LP-SVR algorithm for processing the time-series data to generate the set of support vectors is disclosed herein.

At step 206, logic 108 analyzes the derived set of support vectors. This analysis determines a first set of time periods within the monitoring period within which a measure of density over time of the support vector time points is below a predetermined threshold. This first set of time periods are periods during which the subject is likely to be asleep. This analysis may also determine a second set of time periods within the monitoring period within which the measure of density over time of the support vector time points is equal to or greater than the predetermined threshold. This second set of time periods are periods during which the subject is likely to be awake.

At step 208, logic 108 outputs at least one of (i) the first set of time periods as periods during which the subject is likely to be asleep, and (ii) the second set of time periods as periods during which the subject is likely to be awake. For example, this output may be presented as displayed data on user interface 122. Alternatively, the output may be transmitted to another device via communication device 124.

Different measures of density over time may be used at step 206. One possible measure of density is to count the number of support vectors within a moving time window having a predetermined duration. For instance, a time t may be considered to fall within the first set of time periods if, during a moving time window [t−Δt, t+Δt], there are less than X support vectors, wherein Δt and X are parameters that may be adjusted to increase or decrease the sensitivity of logic 108 in detecting periods of sleep. Conversely, a time t may be considered to fall within the second set of time periods if, during the moving time window [t−Δt, t+Δt], there are equal to or greater than X support vectors. From the perspective of entropy analysis, the density of support vectors within a certain time window is correlated with the entropy of the time-series movement data within that time window. Said another way, a time window having a relatively high density of support vectors indicates that the time-series movement data exhibits relatively high signal variability within that time window. This high signal variability in turn indicates that the subject may be awake during that time window. Conversely, a time window having a relatively low density of support vectors indicates that the time-series movement data exhibits relatively low signal variability within that time window. This low signal variability in turn indicates that the subject may be asleep during that time window.

FIG. 3 is a flowchart depicting an exemplary method 300 for processing raw accelerometer data into movement data suitable for processing by method 200. Method 300 may be implemented by processor 106 of computing device 102, by processor 128 of movement sensor 114, or by both processors 106, 128 working in concert. At step 302, the processor(s) receives raw accelerometer data, such as from the accelerometer 130. At step 304, the processor(s) passes the raw accelerometer data through a low pass filter that removes high-frequency noise. At step 306, the processor(s) passes the output of step 304 through a high pass filter that removes the impact of gravity on accelerometer measurements. At step 308, the processor(s) passes the output of step 306 through a moving average (MA) smoothing filter, which can be used for smoothing the filtered data. And at step 310, the processor(s) provides the output of step 308 as the movement data that is processed by process 200.

An exemplary advantage of the disclosed logic for detecting awake/asleep time periods over prior-known methods of distinguishing between awake/asleep time periods is that it is not reliant upon counting activity/movement within the monitoring period. Rather, by processing the time-series movement data using a LP-SVR algorithm and then evaluating a measure of density over time of the derived set of support vectors, the disclosed logic mitigates the impact of subject-to-subject variations in bodily movement while awake or sleeping (or attempting to sleep).

### Examples

In one example, methods 200 and 300 were tested on a publicly available benchmark dataset published by the Ubiquitous Computing group, part of the Schools for Economic Disciplines and Science and Technology at the University of Siegen in Germany (“Towards a Benchmark for Wearable Sleep Analysis with Inertial Wrist-worn Sensing Units”, Marko Borazio, Eugen Berli, Nagihan Kücükyildiz, Philipp M. Scholl and Kristof Van Laerhoven, ICHI 2014. IEEE Press). The dataset was derived from an open source wrist-worn data logger that contains relatively high-frequency (100 Hz) 3D inertial data from 42 sleep lab patients, along with their data from clinical polysomnography (PSG).

Because the data contained in this public dataset was collected by sampling the activity signals at the relatively high frequency of 100 Hz, the dataset included 3000 activity data points for each 30 second time epoch. To reduce the computational cost and memory burden, the dataset was first down-sampled for preprocessing in this example by selecting only one data point every 10 seconds. The resulting down-sampled dataset was then processed using methods 200 and 300.

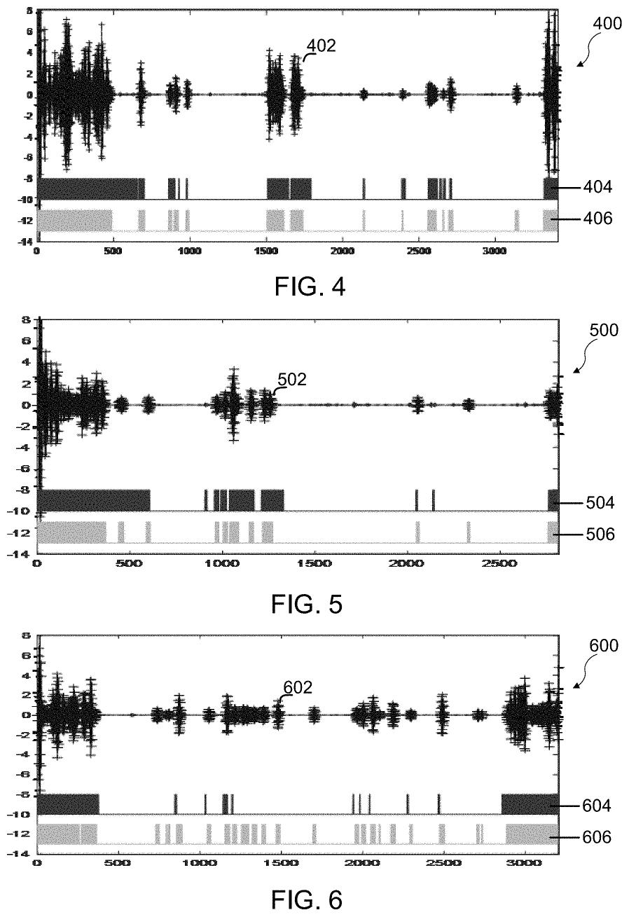

FIGS. 4, 5, and 6 demonstrate the results 400, 500, and 600 obtained by the disclosed methods 200 and 300. Each figure shows the passage of time during the monitored period along the horizontal axis—each datapoint on the horizontal axis corresponds to the passage of 10 seconds, since the public dataset was down-sampled to one datapoint every 10 seconds. Three parts are shown from top to bottom in each figure. The first part (402, 502, 602) is filtered activity data, wherein the vertical displacement of the activity data indicates the magnitude of observed activity during the corresponding time. Plus symbols are used to denote the recorded time-series movement data at the selected support vectors from the SVR algorithm. The second part (404, 504, 604) is the sleep/awake status given by the PSG signal (which was regarded as the ground truth in this example) and the third part (406, 506, 606) is the sleep/awake status identified by the described logic 200 and 300. In the second part (404, 504, 604) and the third part (406, 506, 606), a binary high signal indicates a period where the subject is awake, and a binary low signal indicates a period where the subject is asleep.

FIG. 4 shows data for subject 007 in the above-referenced dataset (who experiences sleep apnea syndrome), while FIGS. 5 and 6 show data for subjects 051 and 030 respectively (who do not experience sleep apnea syndrome). The sleep/awake status identified by the described methods 200 and 300 agreed with the sleep/awake status given by the PSG signal for 87.2% of the monitoring period for results 400 (FIG. 4), for 85% of the monitoring period for results 500 (FIG. 5), and for 80.7% of the monitoring period for results 600 (FIG. 6). The results also achieved a model sparsity (e.g., the ratio of the number of selected support vectors to the number of movement data samples) of 18.7% for results 400 (FIG. 4), of 13.4% for results 500 (FIG. 5), and of 18.7% for results 600 (FIG. 6).

### Exemplary Linear Programming Support Vector Regression (LP-SVR) Algorithm

The Linear Programming Support Vector Regression (LP-SVR) algorithm for use in step 204 of method 200 is presented herein. Compared to the conventional Quadratic Programming Support Vector Regression (QP-SVR) algorithm, the LP-SVR is superior in model sparsity and computational tractability. Consider the nonlinear regression problem in the following:

ƒ(x)=w^{T}φ(x)+b (1)

given training data, {(x₁,y₁), . . . , (x [symbol] ,y [symbol] )} where [symbol] denotes the total number of exemplars, xᵢ∈Rⁿ are the input and yᵢ∈R are the target output data. In the case of sleep/wake detection, xᵢ∈R (i.e., n=1) and denotes the times at which subject activity is recorded by movement sensor 114, and yᵢ∈R denotes the subject activity measured at time i. The nonlinear mapping φ: Rⁿ→R^{m} (m>n) maps the input data into a so-called high dimensional feature space (which can be infinite dimensional) and w∈R^{m}, b∈R. Both LP-SVR and QP-SVR attempt to find a function ƒ(x) that minimizes deviations that are greater than ε from the actually obtained targets yᵢ for all the training data. In finding the function ƒ(x), QP-SVR can be considered to emphasize the smoothness of the model, while LP-SVR emphasizes the sparsity of ƒ(x) rather than the smoothness.

This goal can be formulated as the following constrained optimization problem:

$$minimize \frac{1}{2} {‖ w ‖}^{2} + C {\sum }_{i = 1}^{ℓ} ( {ξ}_{i} + {ξ}_{i}^{*} ) , \\ subject to \{ \begin{matrix}{y}_{i} - 〈 w , φ ( {x}_{i} ) 〉 - b \leq ɛ + {ξ}_{i} \\ 〈 w , φ ( {x}_{i} ) 〉 + b - {y}_{i} \leq ɛ + {ξ}_{i}^{*} \\ {ξ}_{i} , {ξ}_{i}^{*} \geq 0\end{matrix} \qquad ( 2 )$$

where the ξᵢ and ξᵢ* are the slack variables, corresponding to the size of the excess positive and negative deviation respectively. This is a classic quadratic optimization problem with inequality constraints, and the optimization criterion penalizes data points whose y—values differ from ƒ(x) by more than ε. The constant C>0 determines the trade-off between the flatness of ƒ and the amount up to which deviations larger than ε are tolerated.

By defining the following ε—insensitivity loss function,

$$L ( {y}_{i} - f ( {x}_{i} ) ) = \{ \begin{matrix}0 , & if \vert {y}_{i} - f ( {x}_{i} ) \vert \leq ɛ \\ \vert {y}_{i} - f ( {x}_{i} ) \vert - ɛ , & otherwise\end{matrix} \qquad ( 3 )$$

the constrained optimization problem (2) is equivalent to the following regularization problem,

$$minimize {R}_{reg} [ f ] = {\sum }_{i = 1}^{ℓ} L ( {y}_{i} - f ( {x}_{i} ) ) + λ {‖ w ‖}^{2} \qquad ( 4 )$$

where ƒ(x) is in the form of (1) and λ∥w∥² is the regularization term. According to the generalized Representer Theorem (which is explained in further detail in R. Herbrich, Learning Kernel Classifiers: Theory and Algorithms, MIT Press, 2002 (pages 47-49), and B. Schölkopf, A. J. Smola, Learning with Kernels, MIT Press, 2002 (pages 89-92), both incorporated by reference herein), the solution to the regularization problem (4) can be written as the following Support Vector (SV) kernel expansion provided k(xᵢ,xᵢ)=1

$$f ( x ) = {\sum }_{i = 1}^{ℓ} {β}_{i} k ( x , {x}_{i} ) \qquad ( 5 )$$

where k(xᵢ,x) is the kernel function. Three exemplary kernel functions are:

Gaussian radial basis function (GRBF) kernel:

$$k ( x , {x}^{′} ) = exp ( \frac{- {‖ x - {x}^{′} ‖}^{2}}{2 {σ}^{2}} ) ; \qquad ( 6 )$$

Polynomial kernel:

k(x,x′)=(1+ [symbol] x,x′ [symbol] )^{q}; (7)

Sigmoid kernel:

k(x,x′)=tanh(α [symbol] x,x′ [symbol] +γ) (8)

where σ, q, α, γ are the adjustable parameters of the above kernel functions.

The conventional QP-SVR algorithm proceeds by minimizing (4). However, LP-SVR differs from QP-SVR in the use of the regularization term. Specifically, defining)

β=[β₁β₂ . . . [symbol] ]^{T}, (9)

the LP-SVR algorithm replaces (4) by using the [symbol] ₁ norm of β as the regularization term, i.e.,

$$minimize {R}_{reg} [ f ] = {\sum }_{i = 1}^{ℓ} L ( {y}_{i} - f ( {x}_{i} ) ) + λ {‖ β ‖}_{1} \qquad ( 10 )$$

where ƒ(x) is in the form of (5) and ∥β∥₁ denotes the [symbol] ₁ norm in coefficient space. The use of the [symbol] ₁ norm of β leads to a sparse kernel expansion model. This regularization problem is equivalent to the following constrained optimization problem

$$\begin{matrix}minimize \frac{1}{2} {‖ β ‖}_{1} + C {\sum }_{i = 1}^{ℓ} ( {ξ}_{i} + {ξ}_{i}^{*} ) , & ( 11 ) \\ subject to \{ \begin{matrix}{y}_{i} - {\sum }_{j = 1}^{ℓ} {β}_{j} k ( {x}_{j} , {x}_{i} ) \leq ɛ + {ξ}_{i} , \\ {\sum }_{j = 1}^{ℓ} {β}_{j} k ( {x}_{j} , {x}_{i} ) - {y}_{i} \leq ɛ + {ξ}_{i}^{*} , \\ {ξ}_{i} , {ξ}_{i}^{*} \geq 0\end{matrix} . & \end{matrix}$$

From the geometric perspective, it can be followed that ξᵢξᵢ*=0 in SV regression (i.e., one of ξᵢ and ξᵢ* is zero). Therefore, it is sufficient to just introduce slack variable ξᵢ in the constrained optimization problem (11). Thus, we arrive at the following formulation of SV regression with fewer slack variables:

$$minimize \frac{1}{2} {‖ β ‖}_{1} + 2 C {\sum }_{i = 1}^{ℓ} {ξ}_{i} , \\ subject to \{ \begin{matrix}{y}_{i} - {\sum }_{j = 1}^{ℓ} {β}_{j} k ( {x}_{j} , {x}_{i} ) \leq ɛ + {ξ}_{i} , \\ {\sum }_{j = 1}^{ℓ} {β}_{j} k ( {x}_{j} , {x}_{i} ) - {y}_{i} \leq ɛ + {ξ}_{i} , \\ {ξ}_{i} , \geq 0\end{matrix} . \qquad ( 12 )$$

To convert the optimization problem above into a linear programming problem, the βᵢ and |βᵢ| can be decomposed uniquely as follows

βᵢ=αᵢ⁺−αᵢ⁻, (13)

where αᵢ⁺, αᵢ⁻≥0. It is worth noting that the decompositions in (13) are unique, i.e., for a given βᵢ there is only one pair (αᵢ⁺, αᵢ⁻) which fulfills both equations. Furthermore, at least one of αᵢ⁺ and αᵢ⁻ must be zero, i.e., αᵢ⁺·αᵢ⁻=0. This is because according to equation (13), it can be followed that:

$$\begin{matrix}{α}_{i}^{+} = \frac{\vert {β}_{i} \vert + {β}_{i}}{2} & ( 14 ) \\ {α}_{i}^{-} = \frac{\vert {β}_{i} \vert - {β}_{i}}{2} & \end{matrix}$$

So, if βᵢ is positive then αᵢ⁻ is zero, if βᵢ is negative then αᵢ⁺ is zero, and if βᵢ=0 then both αᵢ⁺ and αᵢ⁻ are zero. In this way, the [symbol] ₁ norm of β can be written as

$${‖ β ‖}_{1} = ( \underbrace{1 , 1 , … , 1}_{ℓ} , \underbrace{1 , 1 , … , 1}_{ℓ} ) ( \begin{matrix}{a}^{+} \\ {a}^{-}\end{matrix} ) \qquad ( 15 )$$

where α⁺=(α₁⁺, α₂⁺, . . . , [symbol] )^{T} and α⁻=(α₁⁻, α₂⁻, . . . , [symbol] )^{T}. Furthermore, the constraints in the formulation (12) can also be written in the following vector form

$$( \begin{matrix}K & - K & - I \\ - K & K & - I\end{matrix} ) \cdot ( \begin{matrix}{α}^{+} \\ {α}^{-} \\ ξ\end{matrix} ) \leq ( \begin{matrix}y + ɛ \\ ɛ - y\end{matrix} ) \qquad ( 16 )$$

where Kᵢⱼ=k(xᵢ, xⱼ), ξ=(ξ₁, ξ₂, . . . , ξᵢ)^{T} and I is [symbol] × [symbol] identity matrix. Thus, the constrained optimization problem (12) can be implemented by the following linear programming problem with the variables

$$minimize {c}^{T} ( \begin{matrix}{α}^{+} \\ {α}^{-} \\ ξ\end{matrix} ) , \\ subject to ( \begin{matrix}K & - K & - I \\ - K & K & - I\end{matrix} ) \cdot ( \begin{matrix}{a}^{+} \\ {a}^{-} \\ ξ\end{matrix} ) \leq ( \begin{matrix}y + ɛ \\ ɛ - y\end{matrix} ) \\ where c = {( \underbrace{1 , 1 , … , 1}_{ℓ} , \underbrace{1 , 1 , … , 1}_{ℓ} , \underbrace{2 C , 2 C , … , 2 C}_{ℓ} )}^{T} . \qquad ( 17 )$$

When the linear programming problem of (17) is solved, the output includes a set of coefficients α⁺=(α₁⁺, α₂⁺, . . . , [symbol] )^{T} and α⁻=(α₁⁻, α₂⁻, . . . , [symbol] )^{T}. From these coefficients α⁺ and α⁻, β=[β₁ β₂ . . . β [symbol] ]^{T} may be determined using (13). And from the determined β=[β₁ β₂ . . . [symbol] ]^{T}, ƒ(x) may be determined using (5). The set of support vectors output from the LP-SVR algorithm is the set of values xⱼ for which the corresponding coefficient βⱼ is non-zero.

The terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

1. A computer-implemented method for determining when a subject is awake and when the subject is asleep, the method comprising:
receiving, at least one processor, time-series movement data for the subject derived from accelerometer data recorded by a movement sensor worn by the subject during a monitoring period, wherein the movement data indicates at least a magnitude of bodily movement of the subject at multiple times during the monitoring period;
processing, by the at least one processor, the movement data using a linear programming support vector regression (LP-SVR) algorithm to derive a set of support vectors, wherein each support vector corresponds to a different time point during the monitoring period;
analyzing, by the at least one processor, the derived set of support vectors to determine at least one of (i) a first set of time periods within the monitoring period within which a measure of density over time of the support vectors is below a predetermined threshold, and (ii) a second set of time periods within the monitoring period within which the measure of density over time of the support vectors is above the predetermined threshold; and
outputting, by the at least one processor, at least one of (i) the first set of time periods as periods during which the subject is likely to be asleep, and (ii) the second set of time periods as periods during which the subject is likely to be awake.

2. The method of claim 1, wherein the measure of density over time of the support vectors comprises a count of the number of support vectors within a moving time window having a predetermined duration.

3. The method of claim 1, wherein the movement data is derived from the accelerometer data by passing the accelerometer data through at least one of a low-pass filter, a high-pass filter, a band-pass filter, and a smoothing filter.

4. The method of claim 1, wherein the movement sensor comprises a wrist-mounted actigraph sensor.

5. The method of claim 1, wherein the at least one processor outputs the at least one of the first set of time periods and the second set of time periods to a user-interface displayed on a visual display.

6. A system for determining when a subject is awake and when the subject is asleep, the system comprising:
a movement sensor configured to be worn by the subject during a monitoring period and to record accelerometer data indicating at least a magnitude of bodily movement of the subject at multiple times during the monitoring period; and
a computing device comprising:
at least one communication device configured to receive the recorded accelerometer data from the movement sensor, and
at least one processor configured to:
process the recorded accelerometer data to derive time-series movement data for the subject;
process the movement data using a linear programming support vector regression (LP-SVR) algorithm to derive a set of support vectors, wherein each support vector corresponds to a different time point during the monitoring period;
analyze the derived set of support vectors to determine at least one of (i) a first set of time periods within the monitoring period within which a measure of density over time of the support vectors is below a predetermined threshold, and (ii) a second set of time periods within the monitoring period within which the measure of density over time of the support vectors is above the predetermined threshold; and
output at least one of (i) the first set of time periods as periods during which the subject is likely to be asleep, and (ii) the second set of time periods as periods during which the subject is likely to be awake.

7. The system of claim 6, wherein the measure of density over time of the support vectors comprises a count of the number of support vectors within a moving time window having a predetermined duration.

8. The system of claim 6, wherein the movement data is derived from the accelerometer data by passing the accelerometer data through at least one of a low-pass filter, a high-pass filter, a band-pass filter, and a smoothing filter.

9. The system of claim 6, wherein the movement sensor comprises a wrist-mounted actigraph sensor.

10. The system of claim 6, wherein the at least one processor is further configured to output the at least one of the first set of time periods and the second set of time periods to a user-interface displayed on a visual display.

11. Non-transitory computer-readable media storing computer-executable instructions for determining when a subject is awake and when the subject is asleep that, when executed by at least one processor, is operable to cause the at least one processor to:
receive time-series movement data for the subject derived from accelerometer data recorded by a movement sensor worn by the subject during a monitoring period, wherein the movement data indicates at least a magnitude of bodily movement of the subject at multiple times during the monitoring period;
process the movement data using a linear programming support vector regression (LP-SVR) algorithm to derive a set of support vectors, wherein each support vector corresponds to a different time point during the monitoring period;
analyze the derived set of support vectors to determine at least one of (i) a first set of time periods within the monitoring period within which a measure of density over time of the support vectors is below a predetermined threshold, and (ii) a second set of time periods within the monitoring period within which the measure of density over time of the support vectors is above the predetermined threshold; and
output at least one of (i) the first set of time periods as periods during which the subject is likely to be asleep, and (ii) the second set of time periods as periods during which the subject is likely to be awake.

12. The method of claim 11, wherein the measure of density over time of the support vectors comprises a count of the number of support vectors within a moving time window having a predetermined duration.

13. The method of claim 11, wherein the movement data is derived from the accelerometer data by passing the accelerometer data through at least one of a low-pass filter, a high-pass filter, a band-pass filter, and a smoothing filter.

14. The method of claim 11, wherein the movement sensor comprises a wrist-mounted actigraph sensor.

15. The method of claim 11, wherein the instructions are further operable to cause the at least one processor to output the at least one of the first set of time periods and the second set of time periods to a user-interface displayed on a visual display.
